(19) United States
(12) Patent Application Publication
(10) Pub. No.: US 2024/0174961 A1
(43) Pub. Date: May 30, 2024

(54) CELL DETACHMENT DEVICE AND CELL DETACHMENT METHOD

(71) Applicant: CANON KABUSHIKI KAISHA, Tokyo (JP)

(72) Inventors: NORIHIRO SUZUKI, Kanagawa (JP); TAKUJI OKAMOTO, Tokyo (JP); TAKAYUKI TSUKIMOTO, Kanagawa (JP)

(21) Appl. No.: 18/488,117

(22) Filed: Oct. 17, 2023

(30) Foreign Application Priority Data
Nov. 30, 2022 (JP) 2022-192059

(51) Int. Cl. C12M 3/06; C12M 1/34

(52) U.S. Cl. CPC C12M 27/16; C12M 41/42

(57) ABSTRACT

Provided is a cell detachment device for detaching a cell on a substrate from the substrate by applying vibration, the cell detachment device including: a vibration member configured to be vibrated by a drive voltage at an amplitude depending on an output value of the drive voltage; a vibration transmission member configured to vibrate by receiving vibration of the vibration member, and transmit the vibration of the vibration member to the substrate; a drive voltage output unit configured to output the drive voltage; an amplitude detection unit configured to detect an amplitude of vibration of the vibration transmission member; and a drive voltage control unit configured to control the output value of the drive voltage, based on the amplitude detected by the amplitude detection unit. A cell detachment method which uses the cell detachment device is also provided.

### BACKGROUND OF THE INVENTION

### Field of the Invention

The present invention relates to a technology for detaching a cell from a culture substrate.

### Description of the Related Art

In a medical field, a cell or the like is sometimes cultured in a culture substrate (hereinafter simply referred to as “substrate” in some places) such as a culture plate or dish (a petri dish) to be used for medical treatment or research and development. The cultured cell may adhere to the culture substrate. In order to collect a sample cell after culturing, the cell accordingly requires to be detached from the culture substrate used for the culturing.

Methods of detaching a cell from a culture substrate include a method in which an enzyme for detachment, a chemical that works on a cell membrane, or the like is added to detach the cell, and a method in which the cell is detached by applying vibration energy to the cell through incidence of acoustic radiation pressure.

With the method of detachment that adds an enzyme for detachment, a chemical that works on a cell membrane, or the like, cell proliferation properties and cell activity drop in some cases due to dissolving of proteins of the cell. The method of detaching a cell from a culture substrate by applying a vibration energy to the cell through incidence of acoustic radiation pressure is accordingly preferred, but tends to be high in cost and technical difficulty level.

In Japanese Patent Application Laid-Open No. 2019-30260, there is disclosed a technology for detaching a cell borne in a vessel by projecting ultrasonic waves to an outer surface of the vessel.

In Japanese Patent Application Laid-Open No. 2019-30260, there is disclosed a configuration capable of varying a frequency and an intensity of ultrasonic waves emitted from an ultrasonic transducer in order to detach a cell well with adaptability to various vessels and cells, thicknesses thereof, and the like. However, there is no mention of a specific method for applying vibration at a stable amplitude in a manner suited to an individual difference of a vessel (substrate) and a state of the substrate such as a relative position of the substrate to the ultrasonic transducer (vibration generator).

### SUMMARY OF THE INVENTION

It is accordingly an object of the present invention to provide a cell detachment device and a cell detachment method that are capable of applying vibration at a stable amplitude in a manner suited to an individual difference and a state of a substrate.

According to one aspect of the present invention, there is provided a cell detachment device for detaching a cell on a substrate from the substrate by applying vibration, the cell detachment device including: a vibration member configured to be vibrated by a drive voltage at an amplitude depending on an output value of the drive voltage; a vibration transmission member configured to vibrate by receiving vibration of the vibration member, and transmit the vibration of the vibration member to the substrate; a drive voltage output unit configured to output the drive voltage; an amplitude detection unit configured to detect an amplitude of vibration of the vibration transmission member; and a drive voltage control unit configured to control the output value of the drive voltage, based on the amplitude detected by the amplitude detection unit.

According to another aspect of the present invention, there is provided a cell detachment method for detaching a cell on a substrate from the substrate by applying vibration, the cell detachment method including: vibrating a vibration member by a drive voltage at an amplitude depending on an output value of the drive voltage; detecting an amplitude of vibration of a vibration transmission member, the vibration transmission member being configured to vibrate by receiving vibration of the vibration member, and transmit the vibration of the vibration member to the substrate; and controlling the output value of the drive voltage based on the detected amplitude of the vibration of the vibration transmission member.

Further features of the present invention will become apparent from the following description of exemplary embodiments with reference to the attached drawings.

### BRIEF DESCRIPTION OF THE DRAWINGS

FIG. 1 is a block diagram for illustrating a configuration of a cell detachment device according to a first embodiment of the present invention.

FIG. 2A is an exploded perspective view for illustrating a configuration example of a vibration application unit.

FIG. 2B is a schematic diagram for illustrating arrangement of a vibration member and an amplitude voltage output unit in the vibration application unit.

FIG. 2C is a schematic diagram for illustrating a usage mode example of the vibration application unit illustrated in FIG. 2A.

FIG. 3A is a graph for showing an example of a waveform of a drive voltage output from a drive voltage output unit.

FIG. 3B is a schematic diagram for illustrating a relationship between frequencies of the drive voltage and amplitudes of substrates.

FIG. 3C is a schematic diagram for illustrating a relationship between frequencies of the drive voltage and amplitudes of the substrates.

FIG. 4A is a graph for showing a relationship between a waveform of the drive voltage and a waveform of an amplitude voltage at a vibration frequency other than a natural vibration frequency of the vibration member.

FIG. 4B is a graph for showing a relationship between the waveform of the drive voltage and the waveform of the amplitude voltage at the natural vibration frequency of the vibration member.

FIG. 4C is a schematic diagram for illustrating the amplitude voltage output from an amplitude voltage output unit and amplitude information generated by an amplitude information generation unit.

FIG. 5 is a flow chart for illustrating an example of operation of the cell detachment device in a cell detachment method according to the first embodiment.

FIG. 6 is a graph for showing changes with time of the frequency of the drive voltage.

FIG. 7 is a block diagram for illustrating a configuration of a cell detachment device according to a second embodiment of the present invention.

FIG. 8A is a schematic diagram for illustrating arrangement of a vibration member in a vibration application unit included in the cell detachment device according to the second embodiment of the present invention.

FIG. 8B is a schematic diagram for illustrating arrangement of a vibration transmission member and a displacement amount detector in the cell detachment device according to the second embodiment of the present invention.

### DESCRIPTION OF THE EMBODIMENTS

In detachment of a cell using vibration such as acoustic radiation pressure, a greater amplitude of a vibration generally means a higher cell detachment rate, but too great an amplitude results in a rapid drop in cell survival rate. For that reason, an optimum amplitude of vibration is determined taking the trade-off relationship between the cell detachment rate and the cell survival rate into consideration. In cell detachment operation using vibration, maintaining a stable amplitude of vibration is accordingly required.

However, an examination by the inventors of the present invention has revealed that the vibration generator as described in Japanese Patent Application Laid-Open No. 2019-30260 or a similar vibration generator sometimes fails to stabilize the amplitude despite being operated under a condition that uses the same drive voltage. The fluctuations in the amplitude of vibration have been suspected to be caused by an individual difference of a culture substrate and a state of the substrate such as a relative position of the culture substrate set in a cell detachment device.

Accordingly, the inventors of the present invention have examined execution of feedback control based on the amplitude of vibration of a vibration generator, and have found out that configurations according to the present invention which are described below enable detachment of a cell by a vibration that has a stable amplitude.

Exemplary embodiments of the present invention are described in detail below with reference to the attached drawings. The same or corresponding elements are denoted by the same reference symbol in the drawings, and descriptions thereof are omitted or simplified in some cases. The cell to be detached may be in a free state as an individual cell, a state in which a plurality of cells adhere to one another, or a state in which cells are in a shape of a sheet (a cell sheet).

### First Embodiment

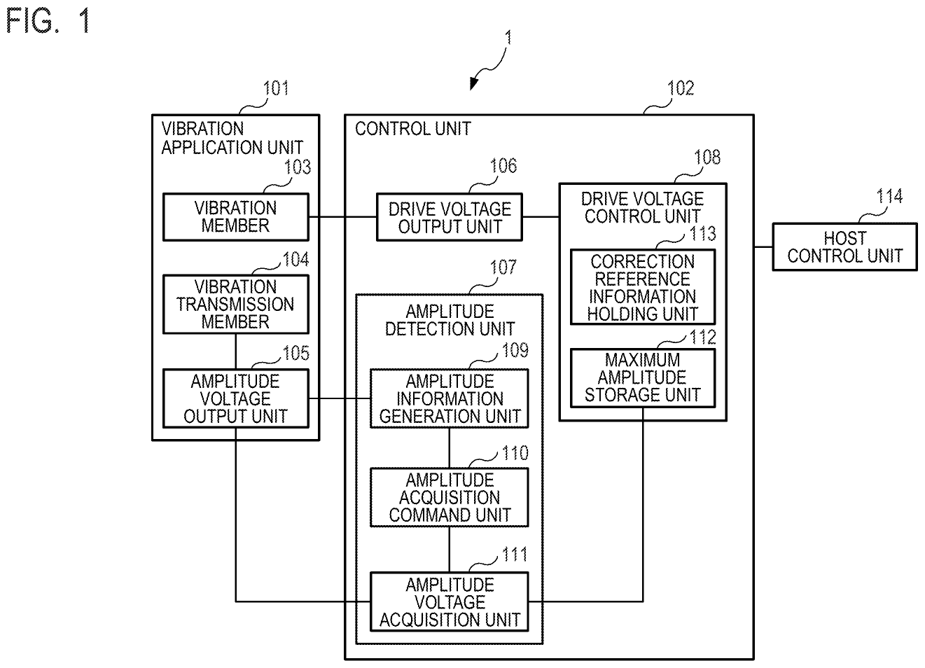

FIG. 1 is a block diagram for illustrating a configuration of a cell detachment device according to the present invention.

A cell detachment device 1 according to the present invention includes a vibration application unit 101 and a control unit 102.

The vibration application unit 101 is a unit for applying vibration to a substrate in order to detach a cell on the substrate, and includes a vibration member 103 and a vibration transmission member 104. The vibration member 103 is a member that is vibrated by a drive voltage at an amplitude depending on an output value of the drive voltage. The vibration transmission member 104 is a member that vibrates by receiving the vibration of the vibration member 103, and transmits the vibration of the vibration member 103 to the substrate. In the first embodiment, the vibration application unit 101 further includes an amplitude voltage output unit 105 which outputs, as a voltage, an amount of displacement of the vibration transmission member 104 that is caused by vibration.

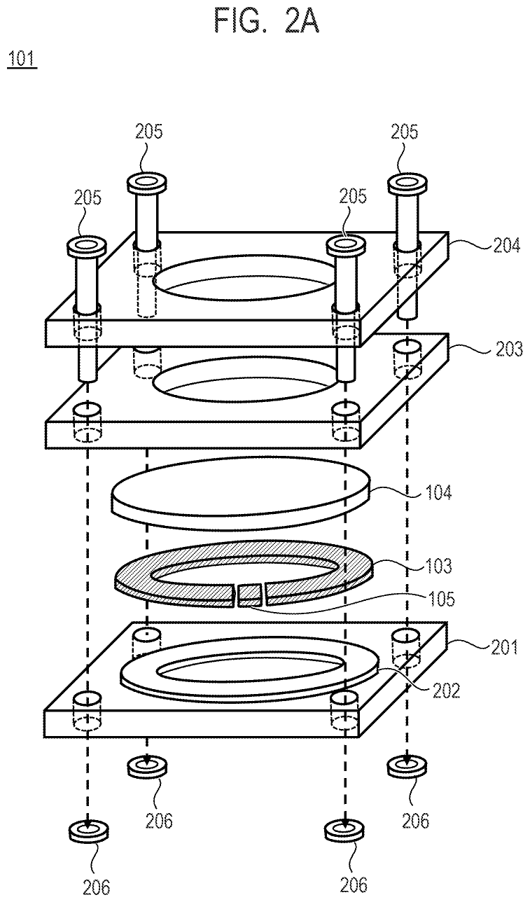

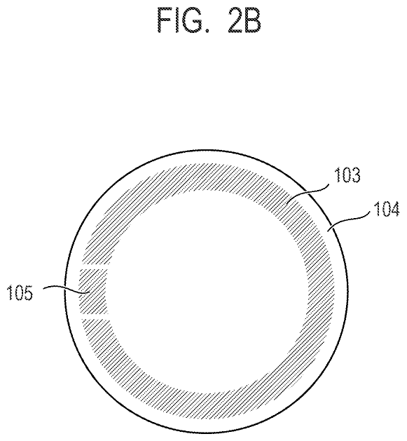

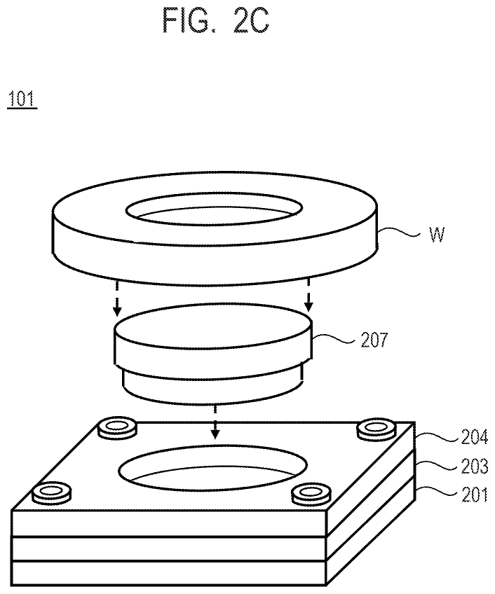

FIG. 2A is illustration of a configuration example of the vibration application unit 101 included in the cell detachment device 1 according to the first embodiment. In FIG. 2A, the vibration application unit 101 is illustrated in an exploded perspective view for the sake of convenience of description.

The vibration application unit 101 illustrated in FIG. 2A includes a lower acrylic plate 201, a felt member 202, the vibration member 103, the amplitude voltage output unit 105, the vibration transmission member 104, an upper silicone rubber member 203, an upper acrylic plate 204, bolts 205, and nuts 206.

The lower acrylic plate 201 is formed from acrylic resin into a square shape, and serves as a base member on which members of the vibration application unit 101 are set. The felt member 202 is formed into a ring shape and is set on the lower acrylic plate 201. The felt member 202 supports the vibration member 103, which is set on the felt member 202, from below. The vibration transmission member 104 is provided on the vibration member 103 and the amplitude voltage output unit 105 in contact with the vibration member 103 and the amplitude voltage output unit 105.

A member made from a material close in vibration transmissibility to a material from which the substrate is made is preferred for the vibration transmission member 104 in order to transmit vibration efficiently to the substrate. For example, a glass plate is usable as the vibration transmission member 104.

FIG. 2B is a schematic diagram for illustrating arrangement of the vibration member 103 and the amplitude voltage output unit 105 in the vibration application unit 101. In the first embodiment, the vibration member 103 includes, among others, an ultrasonic transducer built from a piezoelectric element, and is formed as a ring-shaped member a part of which is cut out for a space to place the amplitude voltage output unit 105 therein. The amplitude voltage output unit 105 is placed in the space provided by cutting out a part of the vibration member 103 in a manner that avoids contact with the vibration member 103. In the first embodiment, the amplitude voltage output unit 105 is a sensor electrode built from a piezoelectric element.

FIG. 2C is illustration of a usage mode example of the vibration application unit 101 illustrated in FIG. 2A. A substrate 207 with a cell to be detached adhering thereto is set on the vibration transmission member 104 through a space provided at a center of the upper acrylic plate 204. A weight W is added on the substrate 207 to hold the substrate 207 in place.

The control unit 102 included in the cell detachment device 1 is a unit for controlling vibration in the vibration application unit 101. The control unit 102 includes a drive voltage output unit 106, an amplitude detection unit 107, and a drive voltage control unit 108. The drive voltage output unit 106 is a function site that outputs a drive voltage for vibrating the vibration member 103. The amplitude detection unit 107 is a function site that detects an amplitude of a vibration of the vibration transmission member 104. The drive voltage control unit 108 is a function site that controls an output value of the drive voltage based on the amplitude detected by the amplitude detection unit 107.

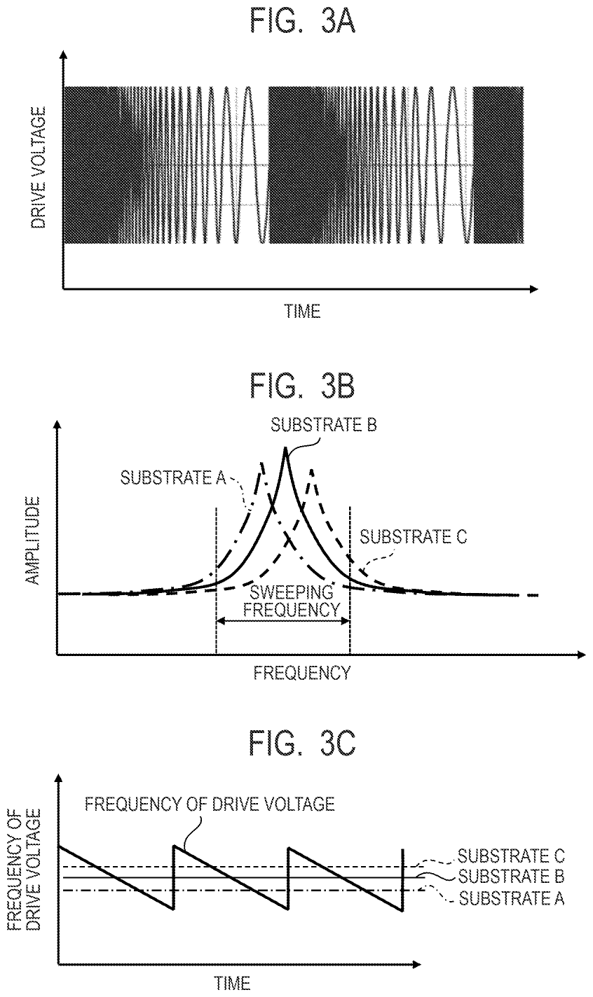

An example of a waveform of the drive voltage output by the drive voltage output unit 106 is shown in FIG. 3A. In the first embodiment, the drive voltage is an alternating voltage, and the drive voltage output unit 106 outputs the drive voltage with a frequency of the drive voltage swept in a range that includes a natural vibration frequency of the vibration member 103. Accordingly, when, for example, the drive voltage is swept from a high frequency side to a low frequency side, changes of the drive voltage become gradual with time in the process of sweeping as shown in FIG. 3A. Once a lower limit value of the sweeping range is reached, the frequency of the drive voltage returns to an upper limit value of the range to be swept toward the lower limit value again, and the drive voltage is output through this repetition.

FIG. 3B and FIG. 3C are each a schematic diagram for illustrating a relationship between frequencies of the drive voltage and amplitudes of the substrates 207.

In FIG. 3B and FIG. 3C, an example in which three exemplary substrates A to C have natural vibration frequencies different from one another due to individual differences or conditions such as arrangement. Despite individual differences and differences in condition such as arrangement among the substrates A to C, the vibration member 103 can be caused to generate a vibration at the natural vibration frequency without fail by sweeping the frequency of the drive voltage.

Although an example of sweeping the frequency of the drive voltage from the upper limit value toward the lower limit value is shown in FIG. 3A to FIG. 3C, the frequency of the drive voltage may be swept from the lower limit value toward the upper limit value, and may also be swept from the upper limit value to the lower limit value and then from the lower limit value to the upper limit value in an alternating manner.

The frequency of the drive voltage is not always required to be swept but, as described above, because vibration can be applied to the substrate at the natural vibration frequency without fail and the cell detachment rate can be improved efficiently, the drive voltage is preferred to be applied with the frequency being swept.

The drive voltage output from the drive voltage output unit 106 is not limited to an alternating voltage. For example, the vibration application unit 101 may be configured so that a digital waveform of a signal such as a Direct Stream Digital (DSD) signal among audio signals is used as a drive waveform, with a low-pass filter placed near the vibration member 103. In this case, the vibration member 103 may include an LC filter structure therein.

In the first embodiment, the amplitude detection unit 107 includes an amplitude information generation unit 109, an amplitude acquisition command unit 110, and an amplitude voltage acquisition unit 111.

The amplitude information generation unit 109 generates amplitude information formed from a rectangular waveform signal. The amplitude information can be generated at timing at which an amplitude voltage output from the amplitude voltage output unit 105 crosses zero, that is, timing at which the amplitude of the vibration of the vibration transmission member 104 reaches 0. To give another example, the amplitude information may be generated at timing at which the drive voltage reaches or drops lower than a predetermined threshold value.

The amplitude acquisition command unit 110 issues a sampling command to sample the amplitude voltage to the amplitude voltage acquisition unit 111, based on the amplitude information generated by the amplitude information generation unit 109. The amplitude voltage acquisition unit 111 acquires a value of the amplitude voltage output from the amplitude voltage output unit 105 in response to the sampling command from the amplitude acquisition command unit 110.

In the first embodiment, the inclusion of the amplitude information generation unit 109, the amplitude acquisition command unit 110, and the amplitude voltage acquisition unit 111 in the amplitude detection unit 107 enables acquisition of a value at a peak of the amplitude of the vibration transmission member 104. A reason therefor is described below.

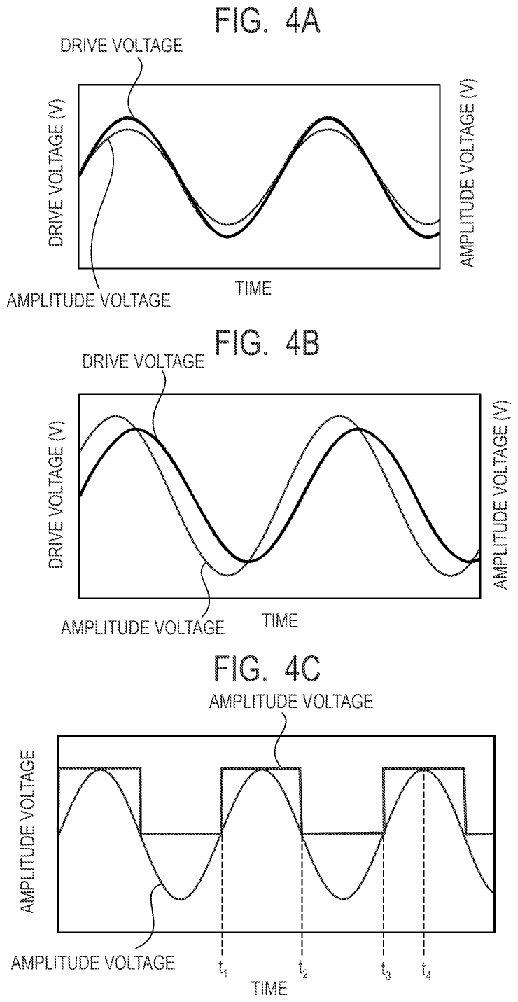

FIG. 4A is a graph for showing a relationship between a waveform of the drive voltage and a waveform of the amplitude voltage at a vibration frequency other than the natural vibration frequency of the vibration member 103. FIG. 4B is a graph for showing a relationship between the waveform of the drive voltage and the waveform of the amplitude voltage at the natural vibration frequency of the vibration member 103. At a vibration frequency other than the natural vibration frequency of the vibration member 103, a phase of the drive voltage and a phase of the vibration of the vibration transmission member 104 match. That is, as shown in FIG. 4A, the phase of the drive voltage and a phase of the amplitude voltage which reflects the vibration of the vibration transmission member 104 match. At the natural vibration frequency of the vibration member 103, on the other hand, there is a gap between the phase of the drive voltage and the phase of the vibration of the vibration transmission member 104.

Consequently, as shown in FIG. 4B, there is a gap between the phase of the drive voltage and the phase of the amplitude voltage which reflects the vibration of the vibration transmission member 104 as well. It is accordingly required to specify sampling timing of the value of the amplitude voltage based on the waveform of the amplitude voltage, not the drive voltage, in order to acquire the value at the peak of the amplitude of the vibration transmission member 104.

FIG. 4C is a schematic diagram for illustrating the amplitude voltage output from the amplitude voltage output unit 105 and the amplitude information generated by the amplitude information generation unit 109.

The amplitude information generation unit 109 generates the amplitude information formed from a rectangular waveform signal as illustrated in FIG. 4C. The amplitude acquisition command unit 110 can predict timing at which the amplitude voltage peaks based on the amplitude information formed from a rectangular waveform signal, and issue a sampling command to sample the amplitude voltage to the amplitude voltage acquisition unit 111. For example, a difference between two successive time points t₁ and t₂ at which the amplitude voltage is 0 is calculated, and, from a time point t₃ at which the amplitude voltage becomes 0 the next time, a time point ta at which the amplitude voltage peaks is predicted to be t₄=t₃+(t₂−t₁)/2. Based on this prediction, the amplitude acquisition command unit 110 can issue a sampling command to sample the amplitude voltage at the time point ta to the amplitude voltage acquisition unit 111. The method of determining the amplitude voltage sampling timing based on the amplitude information is not limited to the example described above, and any method is usable for the determination as long as a value around the peak of the amplitude voltage can be acquired.

The method of acquiring the output value at the peak of the amplitude voltage is not limited to the above-mentioned method which uses the configuration including the amplitude information generation unit 109, the amplitude acquisition command unit 110, and the amplitude voltage acquisition unit 111.

In the first embodiment, the drive voltage control unit 108 includes a maximum amplitude storage unit 112. The maximum amplitude storage unit 112 stores the value of the amplitude voltage acquired by the amplitude voltage acquisition unit 111.

The drive voltage control unit 108 in the first embodiment also includes a correction reference information holding unit 113. The correction reference information holding unit 113 holds correction reference information acquired, in advance, with respect to a relationship between the drive voltage and the amplitude of the vibration of the vibration transmission member 104 which is caused by the drive voltage. For example, the correction reference information can be acquired as follows. First, a displacement gauge (not shown) capable of measuring an amplitude at a center of the vibration transmission member 104 is prepared, and the amplitude at the center of the vibration transmission member 104 is measured at a certain drive voltage. Meanwhile, an amplitude voltage output from the amplitude voltage output unit 105 at the same drive voltage is measured. The amplitude at the center of the vibration transmission member 104 and the amplitude voltage output from the amplitude voltage output unit 105 are then associated with each other, with the drive voltage varied, to thereby acquire the correction reference information. That is, the amplitude voltage output from the amplitude voltage output unit 105 can be converted into the amplitude at the center of the vibration member 103 by using the correction reference information. The drive voltage and the amplitude at the center of the vibration transmission member 104 are in a linear relationship, and the correction reference information may include a slope in the linear relationship. In the first embodiment, the drive voltage control unit 108 controls the output value of the drive voltage with use of the correction reference information held by the correction reference information holding unit 113.

In the first embodiment, the cell detachment device 1 further includes a host control unit 114. The host control unit 114 includes drive command information for driving the control unit 102.

The control unit 102 and the host control unit 114 are configurable from such devices as a personal computer (PC) and a programmable logic controller (PLC).

A cell detachment method according to the first embodiment which uses the cell detachment device 1 is described next.

In the cell detachment method according to the first embodiment, the substrate 207 with a cell to be detached adhering thereto is arranged relative to the cell detachment device 1 as illustrated in FIG. 2C, and the cell is detached by driving the cell detachment device 1.

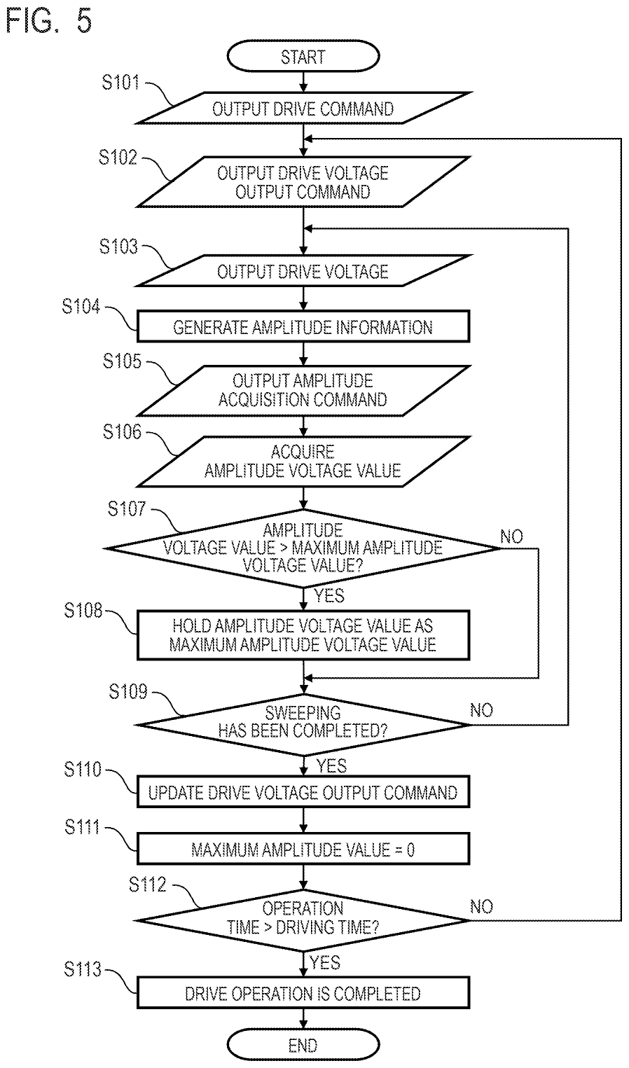

An example of operation of the cell detachment device 1 in the cell detachment method according to the first embodiment is illustrated in FIG. 5.

First, in Step S101, the host control unit 114 outputs a drive command based on the drive command information to the control unit 102.

The drive command information included in the host control unit 114 includes a goal amplitude of the vibration of the vibration transmission member 104 and a driving time. In the example illustrated in FIG. 5, the drive command information further includes the upper limit value and the lower limit value of the sweeping frequency of the drive voltage, and a cycle time (the length of time per sweeping) in which the frequency of the drive voltage is swept. For example, the upper limit value of the sweeping frequency of the drive voltage is 30 kHz, the lower limit value thereof is 20 kHz, the cycle time in which the frequency of the drive voltage is swept is 1 second, and the driving time is 1 minute.

Next, in Step S102, the drive voltage control unit 108 outputs a drive voltage output command to the drive voltage output unit 106, based on the drive command information. The drive voltage output command includes a command about the output value of the drive voltage output from the drive voltage output unit 106. The drive voltage control unit 108 determines the output value of the drive voltage to be output from the drive voltage output unit 106, based on the goal amplitude of the vibration of the vibration transmission member 104 which is included in the drive command information, and a maximum value of the amplitude voltage which is stored in the maximum amplitude storage unit 112. At the start of the driving, however, the maximum amplitude storage unit 112 has not acquired the amplitude voltage, and the drive voltage control unit 108 accordingly determines the output value of the drive voltage by assuming the amplitude voltage stored in the maximum amplitude storage unit 112 to be 0.

Subsequently, in Step S103, the drive voltage output unit 106 outputs the drive voltage with the frequency swept from the upper limit value to the lower limit value, based on the drive voltage output command, and the drive voltage causes the vibration member 103 to vibrate at an amplitude depending on the output value of the drive voltage.

In the first embodiment, the drive voltage output unit 106 outputs the drive voltage with the frequency of the drive voltage swept over a range that includes the natural vibration frequency of the vibration member 103. Here, a case in which the frequency of the drive voltage is swept from the upper limit value toward the lower limit value is described.

Application of the drive voltage to the vibration member 103 causes the vibration member 103 to vibrate, and the vibration transmission member 104 vibrates by receiving the vibration of the vibration member 103. The sensor electrode serving as the amplitude voltage output unit 105 then outputs an amplitude voltage depending on the amplitude of the vibration of the vibration transmission member 104.

In Step S104 to Step S106, the amplitude detection unit 107 detects the maximum value of the amplitude per frequency sweeping, based on the amplitude voltage output from the amplitude voltage output unit 105.

Specifically, in Step S104, the amplitude information generation unit 109 first generates the amplitude information formed from a rectangular waveform signal as illustrated in FIG. 4C, based on the amplitude voltage output from the amplitude voltage output unit 105. Here, the amplitude information generation unit 109 generates the amplitude information at timing at which the amplitude of the vibration of the vibration transmission member 104 becomes 0.

Subsequently, in Step S105, the amplitude acquisition command unit 110 outputs, to the amplitude voltage acquisition unit 111, a sampling command for acquiring the amplitude of the vibration of the vibration transmission member 104 based on the amplitude information generated by the amplitude information generation unit 109 as described above.

Then, in Step S106, the amplitude voltage acquisition unit 111 acquires the value of the amplitude voltage output from the amplitude voltage output unit 105 based on the sampling command from the amplitude acquisition command unit 110.

The value of the amplitude voltage acquired by the amplitude voltage acquisition unit 111 is output to the maximum amplitude storage unit 112. In Step S107, the maximum amplitude storage unit 112 determines whether the value of the amplitude voltage output from the amplitude voltage acquisition unit 111 is a value larger than a value (0 at the start of the driving) of the maximum amplitude voltage already held therein.

When the value of the amplitude voltage acquired from the amplitude voltage acquisition unit 111 is larger than the value of the maximum amplitude voltage already held, the maximum amplitude storage unit 112 holds, in Step S108, the value of the amplitude voltage output from the amplitude voltage acquisition unit 111 as the value of a new maximum amplitude voltage. For example, the maximum amplitude storage unit 112 holds one amplitude voltage value as the maximum amplitude voltage value and, when the amplitude detection unit 107 detects an amplitude voltage value larger than the held amplitude voltage value, may update the held amplitude voltage value.

When it is determined in Step S107 that the value of the amplitude voltage output from the amplitude voltage acquisition unit 111 is not a value larger than the value of the maximum amplitude voltage already held, Step S108 is skipped and operation of Step S109 is executed.

Subsequently, in Step S109, the drive voltage control unit 108 determines whether sweeping of the frequency of the drive voltage has been completed. When it is determined that the sweeping has not been completed, the process returns to the operation of Step S104.

When it is determined in Step S109 that the sweeping has been completed, the drive voltage control unit 108 updates the drive voltage output command in Step S110. Specifically, the drive voltage control unit 108 determines a new output value of the drive voltage with use of the correction reference information, based on the goal amplitude of the vibration of the vibration transmission member 104 which is included in the drive command information and on the maximum amplitude voltage value.

Then, in Step S111, the maximum amplitude storage unit 112 initializes the held maximum amplitude voltage value to 0.

In Step S112, the drive voltage control unit 108 determines whether a time elapsed since the start of the driving (regarded as an operation time) has exceeded the driving time included in the drive command information.

When it is determined that the operation time has not exceeded the driving time, the process returns to Step S102, and the drive voltage control unit 108 outputs the updated drive voltage output command to the drive voltage output unit 106. The drive voltage output unit 106 outputs, with the frequency swept from the upper limit value to the lower limit value again, a drive voltage based on the drive voltage output command updated by the drive voltage control unit 108.

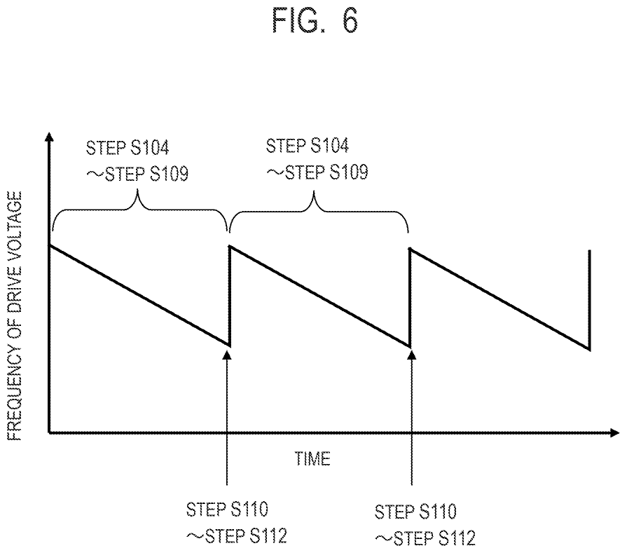

Changes with time of the frequency of the drive voltage in a period from Step S104 to Step S112 are shown in FIG. 6.

As shown in FIG. 6, from Step S104 to Step S109, the frequency of the drive voltage is swept from the upper limit value toward the lower limit value, and becomes lower with time. When the process returns to Step S102 after Step S110 to Step S112, the frequency of the drive voltage is raised to the upper limit value. Then, at the output value of the drive voltage updated in Step S110, the frequency is again swept from the upper limit value toward the lower limit value, and the operation of Step S103 to Step S109 is executed once more.

When it is determined in Step S112 that the operation time has exceeded the driving time, the drive voltage control unit 108 updates, in Step S113, the drive voltage output command so that the drive voltage output unit 106 stops outputting the drive voltage, and drive operation of the cell detachment device 1 is completed.

In the first embodiment, by driving the cell detachment device 1 in the manner described above, not only effective detachment by vibration at the natural vibration frequency but also detachment that maintains a desired amplitude through the control described above is accomplished. This enables cell detachment that steadily maintains a desired condition determined based on the cell detachment rate and the cell survival rate.

### Second Embodiment

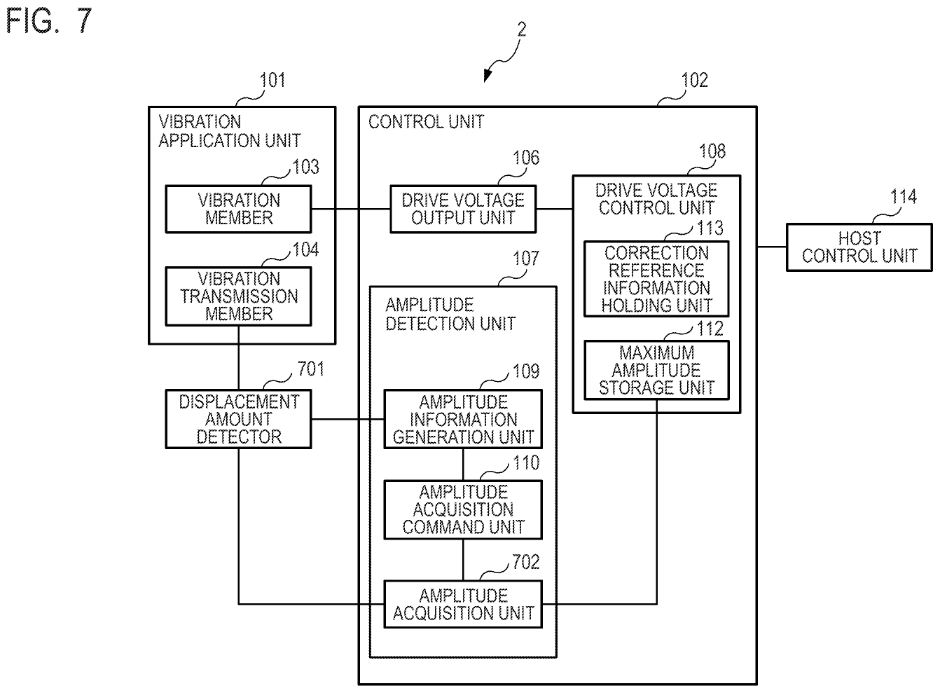

FIG. 7 is a block diagram for illustrating a configuration of a cell detachment device 2 according to a second embodiment of the present invention.

The cell detachment device 2 includes, in place of the amplitude voltage output unit 105 in the cell detachment device 1 according to the first embodiment, a displacement amount detector 701, which is an independent component separate from the vibration application unit 101. The cell detachment device 2 also includes an amplitude acquisition unit 702 in place of the amplitude voltage acquisition unit 111 in the cell detachment device 1 according to the first embodiment.

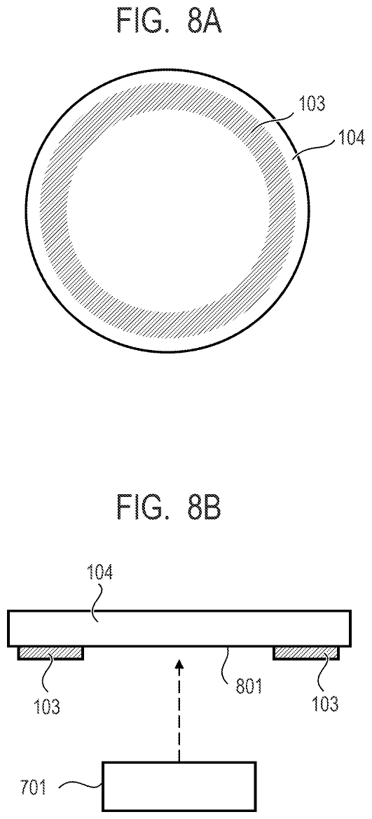

FIG. 8A is a schematic diagram for illustrating arrangement of the vibration member 103 in the vibration application unit 101 included in the cell detachment device 2 according to the second embodiment. FIG. 8B is a schematic diagram for illustrating arrangement of the vibration transmission member 104 and the displacement amount detector 701 in the cell detachment device 2 according to the second embodiment. In FIG. 8B, a positional relationship between the vibration transmission member 104 and the displacement amount detector 701 viewed from a direction perpendicular to a direction in which the vibration member 103 and the vibration transmission member 104 are aligned is illustrated.

Unlike the cell detachment device 1, the cell detachment device 2 does not include the amplitude voltage output unit 105, and the vibration member 103 accordingly has a ring shape without a cut-out part as illustrated in FIG. 8A.

As illustrated in FIG. 8B, in place of the amplitude voltage output unit 105, the cell detachment device 2 has a laser displacement gauge as the displacement amount detector 701 at a predetermined distance from the center of the vibration transmission member 104 in a direction perpendicular to a vibration surface 801 of the vibration transmission member 104.

In the second embodiment, the vibration of the vibration transmission member 104 caused by the vibration of the vibration member 103 is detected by the laser displacement gauge serving as the displacement amount detector 701. The displacement amount detector 701 detects a displacement amount at a central portion of the vibration transmission member 104, and outputs an analog signal depending on magnitude of the displacement amount.

In the second embodiment, in Step S104 described in the cell detachment method according to the first embodiment, the amplitude information generation unit 109 generates amplitude information formed from a rectangular waveform signal as illustrated in FIG. 4C, based on the analog signal described above. Subsequently, in Step S105, the amplitude acquisition command unit 110 outputs, based on the amplitude information, a command to acquire the amplitude of the vibration of the vibration transmission member 104. Then, in Step S106, the amplitude acquisition unit 702 acquires a value of the displacement amount output from the displacement amount detector 701, based on the sampling command from the amplitude acquisition command unit 110. In the second embodiment, the maximum amplitude storage unit 112 is configured to store the value of the displacement amount instead of the amplitude voltage.

Operation of the cell detachment device 2 according to the second embodiment is basically the same as the operation of the cell detachment device 1 according to the first embodiment, except that, as described above, the control unit 102 operates based on the analog signal output from the displacement amount detector 701.

The displacement amount detector 701 in the second embodiment may be a measurement instrument other than a laser displacement gauge that is based on another method capable of measuring the displacement amount at the center of the vibration transmission member 104, and is not limited to any instrument. For example, an electrode may be provided in parallel to the vibration member 103 and the vibration transmission member 104 to detect changes in electrostatic capacitance, or an acceleration sensor may be provided in the vibration transmission member 104.

According to the present invention, it is possible to provide the cell detachment device and the cell detachment method that are capable of applying vibration at a stable amplitude in a manner suited to an individual difference and a state of a substrate.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2022-192059, filed Nov. 30, 2022, which is hereby incorporated by reference herein in its entirety.

What is claimed is:

1. A cell detachment device for detaching a cell on a substrate from the substrate by applying vibration, the cell detachment device comprising:
a vibration member configured to be vibrated by a drive voltage at an amplitude depending on an output value of the drive voltage;
a vibration transmission member configured to vibrate by receiving vibration of the vibration member, and transmit the vibration of the vibration member to the substrate;
a drive voltage output unit configured to output the drive voltage;
an amplitude detection unit configured to detect an amplitude of vibration of the vibration transmission member; and
a drive voltage control unit configured to control the output value of the drive voltage, based on the amplitude detected by the amplitude detection unit.

2. The cell detachment device according to claim 1,
wherein the drive voltage is an alternating voltage, and
wherein the drive voltage output unit is configured to output the drive voltage with a frequency of the drive voltage swept over a range that includes a natural vibration frequency of the vibration member.

3. The cell detachment device according to claim 2,
wherein the drive voltage control unit includes a maximum amplitude storage unit configured to store a maximum value of the amplitude of the vibration transmission member per sweeping of the frequency, and
wherein the drive voltage control unit is configured to control the output value of the drive voltage based on the maximum value of the amplitude of the vibration transmission member.

4. The cell detachment device according to claim 3, wherein the amplitude detection unit is configured to detect the maximum value of the amplitude per sweeping of the frequency.

5. The cell detachment device according to claim 4, wherein the amplitude detection unit includes:
an amplitude information generation unit configured to generate amplitude information formed from a rectangular waveform signal;
an amplitude acquisition command unit configured to output, based on the amplitude information, a command to acquire the amplitude of the vibration of the vibration transmission member; and
an amplitude acquisition unit configured to acquire the amplitude of the vibration of the vibration transmission member, based on the command output from the amplitude acquisition command unit.

6. The cell detachment device according to claim 1,
wherein the drive voltage control unit includes a correction reference information holding unit configured to hold correction reference information acquired in advance with respect to a relationship between the drive voltage and the amplitude of the vibration of the vibration transmission member that is caused by the drive voltage, and
wherein the drive voltage control unit is configured to control the output value of the drive voltage with use of the correction reference information.

7. The cell detachment device according to claim 1, further comprising a sensor electrode which is a piezoelectric element provided in contact with the vibration transmission member,
wherein the amplitude detection unit is configured to detect the amplitude of the vibration of the vibration transmission member based on an amplitude voltage output from the sensor electrode.

8. A cell detachment method for detaching a cell on a substrate from the substrate by applying vibration, the cell detachment method comprising:
vibrating a vibration member by a drive voltage at an amplitude depending on an output value of the drive voltage;
detecting an amplitude of vibration of a vibration transmission member, the vibration transmission member being configured to vibrate by receiving vibration of the vibration member, and transmit the vibration of the vibration member to the substrate; and
controlling the output value of the drive voltage based on the detected amplitude of the vibration of the vibration transmission member.

9. The cell detachment method according to claim 8,
wherein the drive voltage is an alternating voltage, and
wherein the cell detachment method further comprises outputting the drive voltage with a frequency of the drive voltage swept over a range that includes a natural vibration frequency of the vibration member.

10. The cell detachment method according to claim 9, further comprising:
storing a maximum value of the amplitude of the vibration transmission member per sweeping of the frequency; and
controlling the output value of the drive voltage based on the maximum value of the amplitude of the vibration transmission member.

11. The cell detachment method according to claim 10, further comprising detecting the maximum value of the amplitude per sweeping of the frequency.

12. The cell detachment method according to claim 11, further comprising:
generating amplitude information formed from a rectangular waveform signal;
outputting, based on the amplitude information, a command to acquire the amplitude of the vibration of the vibration transmission member; and
acquiring the amplitude of the vibration of the vibration transmission member, based on the command to acquire the amplitude of the vibration of the vibration transmission member.

13. The cell detachment method according to claim 8, further comprising controlling the output value of the drive voltage with use of correction reference information acquired in advance with respect to a relationship between the drive voltage and the amplitude of the vibration of the vibration transmission member that is caused by the drive voltage.

14. The cell detachment method according to claim 8, further comprising detecting the amplitude of the vibration of the vibration transmission member based on an amplitude voltage output from a sensor electrode which is a piezoelectric element provided in contact with the vibration transmission member.
